(19) United States
(12) Patent Application Publication
(10) Pub. No.: US 2003/0228999 A1
(43) Pub. Date: Dec. 11, 2003

(54) Method, an apparatus, and a composition for customizing the combination of fragrances

(75) Inventors: Don Roderick Donovan, Sebastopol, CA (US); Jane Margaret Warwick, Frimley (GB); Robert David Piotrowski, Hamilton, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH

(21) Appl. No.: 10/339,143

(22) Filed: Jan. 9, 2003

Related U.S. Application Data
(63) Continuation of application No. PCT/US01/21773, filed on Jul. 10, 2001.
(60) Provisional application No. 60/217,672, filed on Jul. 10, 2000.

(51) Int. Cl. A61K 7/46

(52) U.S. Cl. 512/1

(57) ABSTRACT

An apparatus and method for preparing a customized fragrance is disclosed. The method comprises: 1) selecting a first fragrance, the first fragrance comprising a base note, a middle note and a top note, 2) selecting a second fragrance, the second fragrance comprising a base note, a middle note and a top note, and 3) combining the first fragrance with the second fragrance to form the customized fragrance.

### CROSS REFERENCE TO RELATED APPLICATION

This is a continuation of International Application PCT/US01/21773, with an international filing date of Jul. 10, 2001 which claims benefit of U.S. Provisional Application Serial No. 60/217,672 filed on Jul. 10, 2000.

### BACKGROUND OF THE INVENTION

1. Field of the Invention

The invention relates to fragrances and more particularly to a method of combining fragrances to form a customized fragrance.

2. Background

Creating fragrances is known in the art. Such fragrances are used in a variety of ways such as perfumes, cologne, soap, shampoo, etc. Perfumes, for example, are made up of an assortment of Perfume Raw Materials (PRMs). The selection of the PRMs to create a perfume is typically performed on a trial and error basis. For example, a manufacturer of a perfume may combine PRM A and PRM B with PRM C and then determine whether the mixture of PRMs is appealing based upon the smell of the combined PRMs. The manufacturer may then test whether the general public finds the perfume to be appealing.

The trial and error method of creating a perfume may create a fragrance which may only be appealing to, for example, 10% or less of the population. Typically, a fragrance is not commercially viable unless at least 10% of the targeted market find it appealing. One disadvantage of trial and error approach is that mass marketed fragrances may not be appealing to a particular individual or a substantial portion of the targeted market.

Some customers may desire customized fragrances suited to their individual requirements. Customizing fragrances to an individual, however, is expensive. In one prior art approach, customers are given an opportunity to select from hundreds of available PRMs. The use of PRMs is well known. Once the various selections are made, the fragrance can be created by mixing the selected PRMs. However, it is common for the resulting fragrance to be unappealing to the consumer. Although this prior art approach gives a consumer complete flexibility in specifying the content or PRMs of the fragrance, the success rate of specifying an appealing fragrance is extremely low.

In another prior art implementation for customizing the production of fragrances, modifications are made to a conventional triangle used to represent the basic components of most all cosmetic fragrances. Such a conventional triangle is illustrated in FIG. 2. It is well known that fragrances are comprised of a combination of base notes 230 (also denoted bottom notes or base components), middle notes 220 (also denoted middle components), and top notes 210 (also denoted top components). Exemplary base notes, middle notes and top notes are discussed in U.S. Pat. No. 5,354,737, the teachings of which are incorporated herein by reference. Base notes are characterized by components with long-lasting character. Musk is one example of a base note. Base notes typically have a higher molecular weight than other notes. Middle notes have a lower molecular weight than base notes, but higher molecular weight than top notes. Top notes are characterized by their high volatility and low molecular weight. Jasmine is one example of a top note. Middle notes 220 of triangle 200, have a molecular weight that is higher than the top notes 210 and lower than the base notes 230, middle notes 220 and base notes 230 provide the longevity of the fragrance, such as the smell after several hours that the fragrance was first placed on an individual's skin. The top notes 210, on the other hand, typically dominate the smell of the fragrance when the fragrance is first placed on the skin of an individual. Because top notes 210 are more volatile than middle and base notes (220, 230), the top notes 210 dissipate more quickly. The molecular weight of the top notes is generally much lower than the molecular weight of the middle and base notes.

Again, as is well known in the art, all conventional cosmetic fragrances can be represented by a fragrance triangle, such as the one illustrated in FIG. 2 and discussed above. In some prior art customization processes, a customer is offered the option of replacing a top note with one of several other optional top notes. Although this approach gives the consumer some fragrance selection options, the options are limited by the small number of appealing fragrance combinations that can be produced.

Accordingly, it is desirable to overcome the known disadvantages related to the conventional methods used to create a customized fragrance.

### SUMMARY

An apparatus and method for preparing a customized fragrance is disclosed. The method comprises: 1) selecting a first fragrance, the first fragrance comprising a base note, a middle note and a top note, 2) selecting a second fragrance, the second fragrance comprising a base note, a middle note and a top note, and 3) combining the first fragrance with the second fragrance to form the customized fragrance. Additional features, embodiments, and benefits will be evident in view of the figures and detailed description presented herein.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1 illustrates a fragrance triangle partitioned into two fragrance areas used to select a first fragrance and a second fragrance in accordance with one embodiment of the invention;

FIG. 2 illustrates a conventional fragrance triangle used for fragrance descriptors in the prior art;

FIG. 3 illustrates one embodiment of a computer system that implements the principles of the present invention;

FIG. 4 illustrates a flow diagram for selecting first and second fragrances to form a customized fragrance in accordance with one embodiment of the invention;

FIG. 5 illustrates a flow diagram for selecting a first fragrance in accordance with one embodiment of the invention; and

FIG. 6 illustrates a flow diagram for selecting a second fragrance in accordance with one embodiment of the invention.

FIG. 7 illustrates a fragrance triangle partitioned into three or more fragrance areas used to select a first fragrance and a second fragrance in accordance with one embodiment of the invention;

FIGS. 8-16 illustrate a user interface in accordance with one embodiment of the invention;

FIGS. 17-34 illustrate various examples of images used to represent fragrances in one embodiment of the invention.

### DETAILED DESCRIPTION

An apparatus and method for preparing a customized fragrance is disclosed. The method comprises: 1) selecting a first fragrance, the first fragrance comprising a base note, a middle note and a top note, 2) selecting a second fragrance, the second fragrance comprising a base note, a middle note and a top note, and 3) combining the first fragrance with the second fragrance to form the customized fragrance. The method of the present invention for selecting first and second fragrances in order to create a customized fragrance can be carried out using any interactive means. For instance, the process can be implemented on a network, such as the Internet, or on a stand-alone computer. Alternatively, this invention may be implemented using a variety of conventional methods such as by telephone, by direct mail, or face-to-face meetings between the consumer and the manufacturer, distributor or beauty consultant. It would be readily apparent to those of ordinary skill in the art that any other means that allow for a dynamic, interactive process can be used to carry out the processes of the present invention.

In the following description, numerous specific details are set forth to provide a thorough understanding of the invention. However, it will be understood by one of ordinary skill in the art that the invention may be practiced without these specific details. In other instances, well known structures and techniques have not been shown in detail to avoid obscuring the invention.

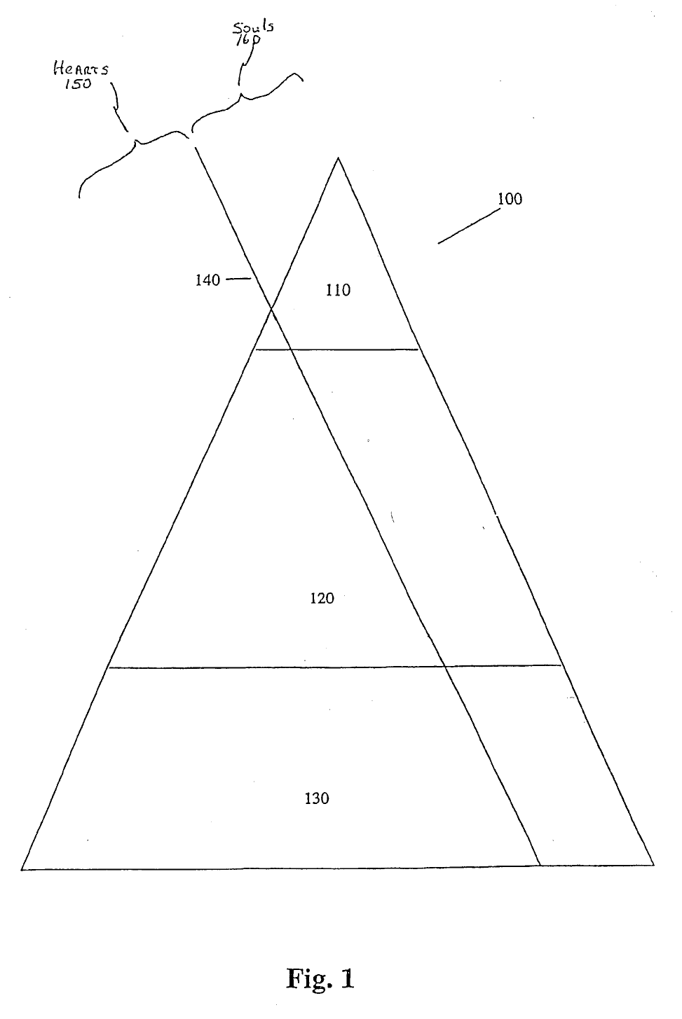

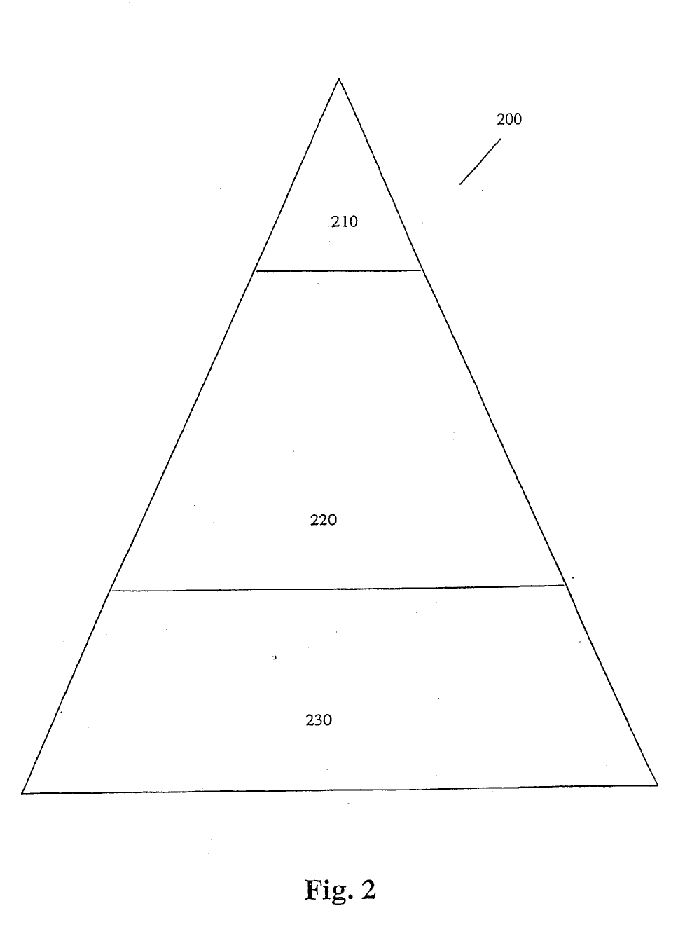

FIG. 1 illustrates, in part, the conventional fragrance triangle 100 partitioned into three areas as discussed above and partitioned diagonally as discussed below.

In accordance with the teachings of the present invention, diagonal line 140 is added to the conventional fragrance triangle. Diagonal line 140 partitions triangle 100 to provide a boundary between the first fragrance 150 and the second fragrance 160. The first fragrance 150 comprises at least one top note 110, at least one middle note 120, and at least one base note 130. In a presently preferred embodiment, the first fragrance 150 is dominated by the base and middle notes (i.e., it comprises no more than 30% (by volume) top note. The second fragrance 160 is also comprised of at least one top note 110, at least one middle note 120, and at least one base note 130. The second fragrance 160 is dominated by the more volatile top note 110 and the middle note 120 (i.e., it comprises 30% (by volume) or more of top note). The second fragrance 160 is also comprised of at least one top note 110, at least one middle note 120, and at least one base note 130. The second fragrance 160 is dominated by the more volatile top note 110 and the middle note 120.

Based on the teaching of the present invention, it will be readily apparent to those of skill in the art that the fragrance triangle illustrated in FIG. 1 can be diagonally partitioned using multiple diagonal links 140, thereby producing a plurality of fragrances, each fragrance having a base note, middle note, and top note, and any number of which can be combined to produce a customized fragrance.

The first fragrance 150 (also denoted herein by the term “hearts”) and the second fragrance 160 (also denoted herein by the term “souls”) can be combined in any ratio. In a preferred embodiment, the ratio of the first fragrance to second fragrance (i.e., hearts:souls) ranges from 90:10 (by volume), more preferably 80:20 (by volume), more preferably 70:30 (by volume), more preferably 60:40 (by volume) and given more preferably 50:50 (by volume).

One advantage of the present invention enabled by the novel fragrance triangle partitioning described above is that both the hearts 150 and the souls 160 represent independently viable fragrances. Because each of the hearts 150 and souls 160 have top, middle, and base notes, each of the hearts 150 and souls 160 can themselves be viable fragrances on their own. Thus, it can be determined through field trials whether individual hearts 150 and souls 160 are appealing fragrances. Using field trials, or other means, a subset of appealing hearts 150 and souls 160 can be identified. Once this set of appealing hearts 150 and souls 160 is identified, these hearts 150 and souls 160 can be used as the set of fragrance combinations from which a consumer may select a particular heart 150 to be combined with a particular soul 160. Because it will already have been determined that individual hearts 150 and souls 160 are independently appealing fragrances, there is a high probability that the combination of a particular heart 150 and a particular soul 160 will also be an appealing combination. The process used in the present invention for enabling a consumer to select a particular heart 150 and a particular soul 160 for combination into a combined customized fragrance is described below.

In order to select the fragrance 150 and the second fragrance 160, an individual, such as a consumer, must respond to a series of questions to determine the consumer's particular tastes. To select a particular first fragrance 150, the individual is required in one embodiment, to respond to four questions. The first question relates to the time of day that the individual expects to be using the fragrance and the effect that fragrance has during the day when it may be at a higher temperature compared to its effect if the individual was wearing the perfume in the evening. The second question relates to the occasion at which the individual intends to wear the fragrance. For example, the individual may be seeking a fragrance that is acceptable in a casual setting as opposed to a very special or more formal setting. The third question relates to the mood that the individual seeks to evoke from others. For example, the mood that the individual may seek to set is a confident mood, a seductive mood, a feminine mood, a calm mood, or an energized mood. The fourth question relates to whether the individual is seeking to provide a traditional or trendy smell. It will be apparent to those of ordinary skill in the art that other queries for determining an individual's tastes or performances may also be employed. Such inquiries may relate to demographics, geographies and/or psychographics and include, for example, past consumer behavior and selections, age, ethnicity, skin-type (oily or dry), location, etc. The computer, evaluator and/or manufacturer will match these responses to determine the selected first fragrance 150. For example, if a woman is seeking to wear the fragrance during the daytime, and she desires to have a trendy effect with an energized mood on a casual occasion, a Marine fragrance would be selected as the first fragrance 150. In yet another example, if the woman is to wear the perfume in the evening and desires a seductive mood for a special occasion and she also desires to be trendy, a Gourmand fragrance would be selected as the first fragrance 150.

After selecting the first fragrance 150, a second fragrance 160 is then selected, based, in part, on the particular first fragrance 150 selected. In particular, the selection of a first fragrance 150 will rank and narrow the available second fragrance 160 to only those that are most highly compatible with the selected first fragrance 150. In this manner, the present invention will produce a combined customized fragrance that is very likely to be appealing to the targeted market. In order to further select the second fragrance 160, in one embodiment, additional questions are posed to the individual. For example, a question relating to where a person wishes to evoke a sense of transportation to a fantasy location, such as Florence, Italy, or other memorable location is asked. Similarly, a question relating to whether the fragrance is going to be worn in a particular season, such as spring, summer, fall, or winter is asked.

For example, if a Marine fragrance is selected as the first fragrance 150, and if a woman has the fantasy to be transported to Venice, Italy in the spring, then Fresh Marine is selected as the second fragrance 160. However, three souls would be selected as being highly compatible with the selected first fragrance 150 (e.g., white flower, fresh marine and green floral), with the white flower being designated as the most preferred choice for the second fragrance 160.

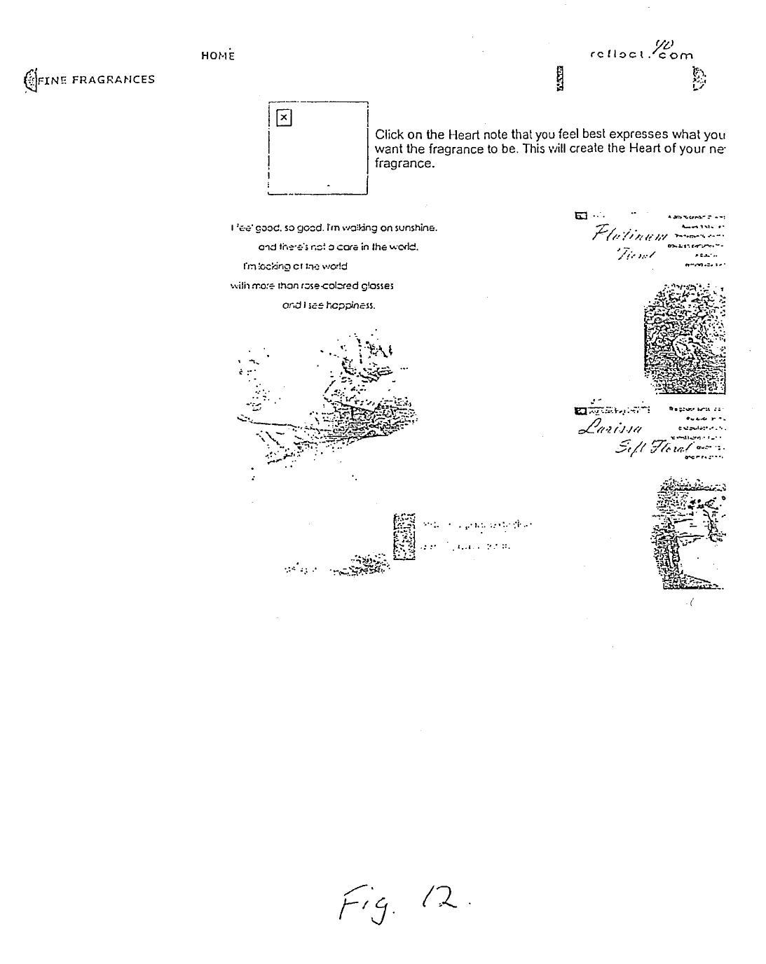

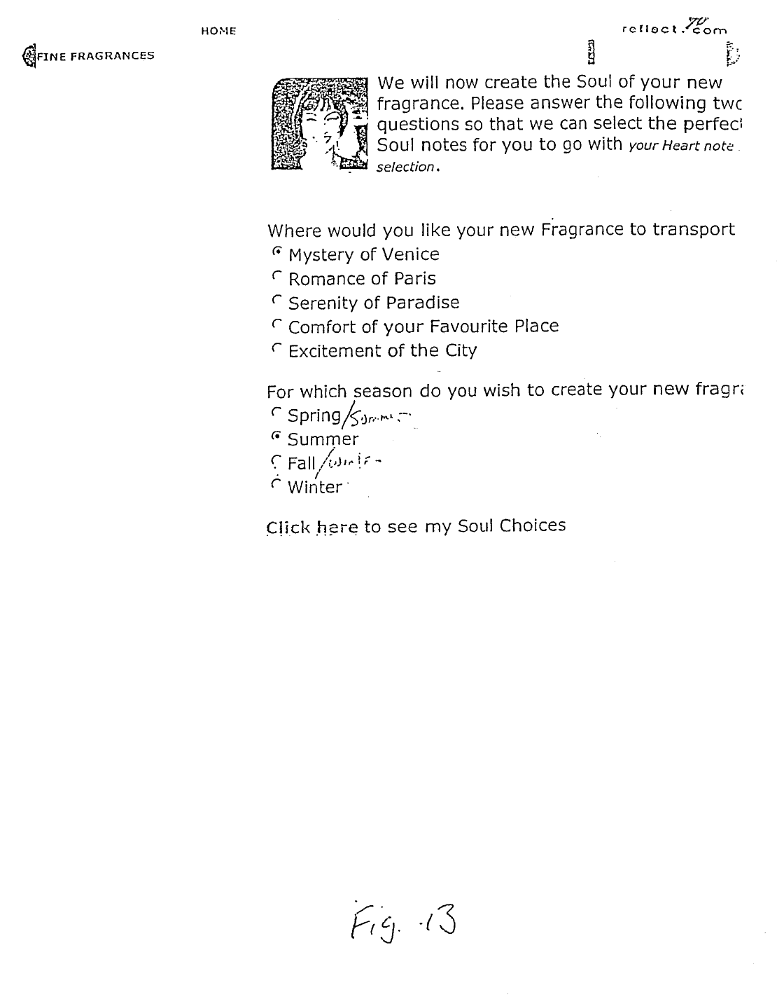

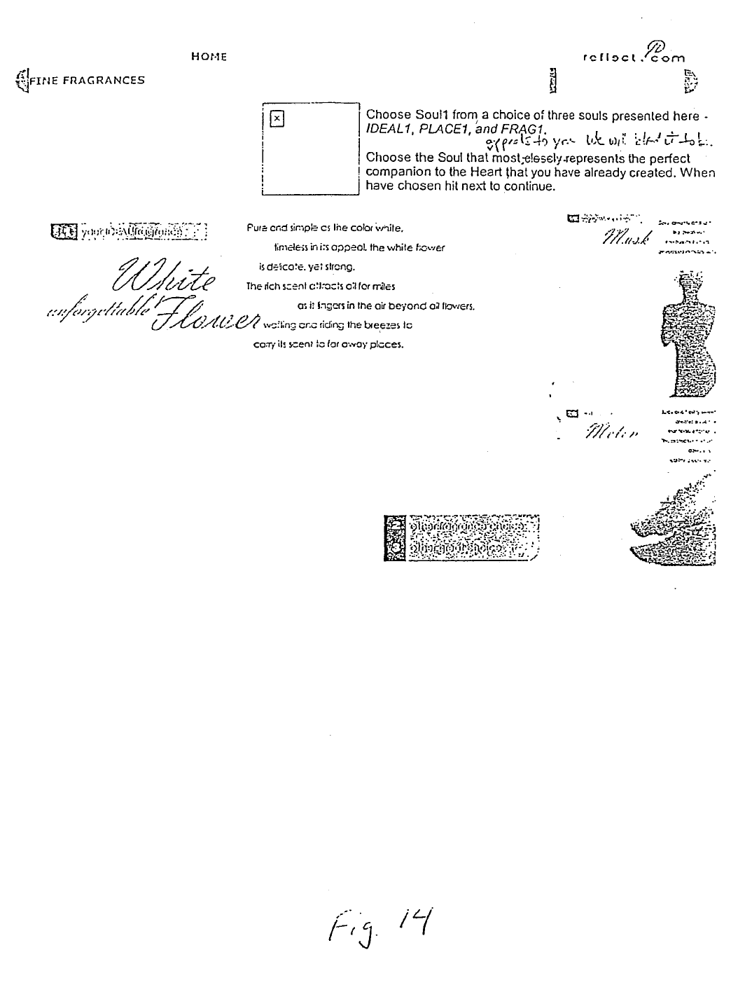

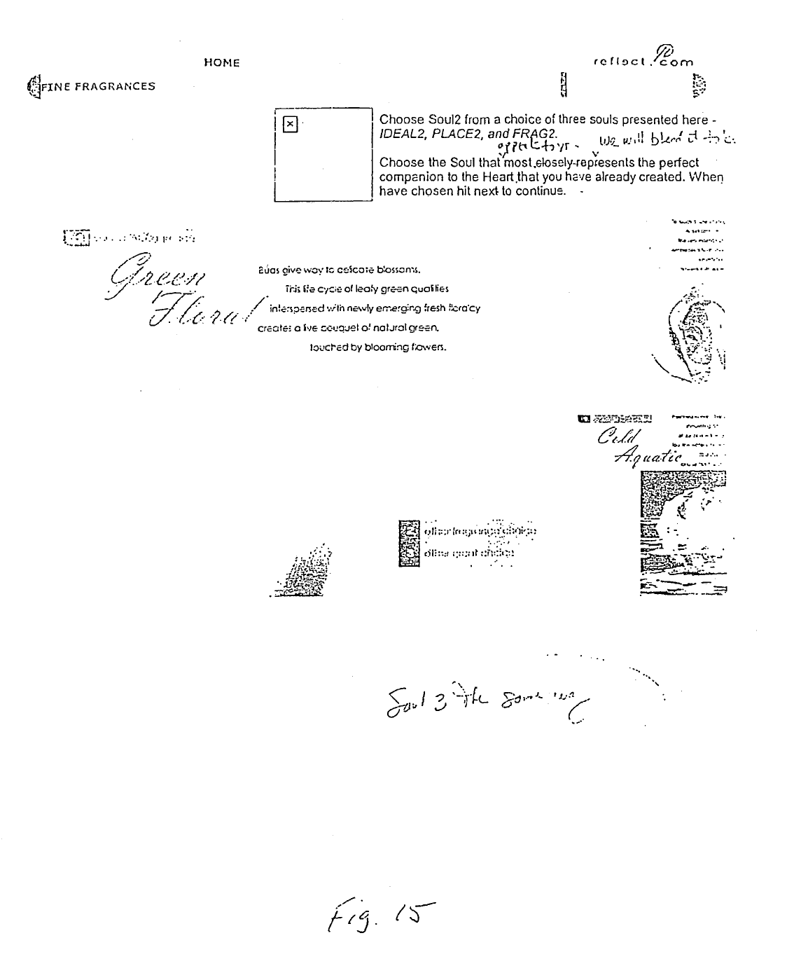

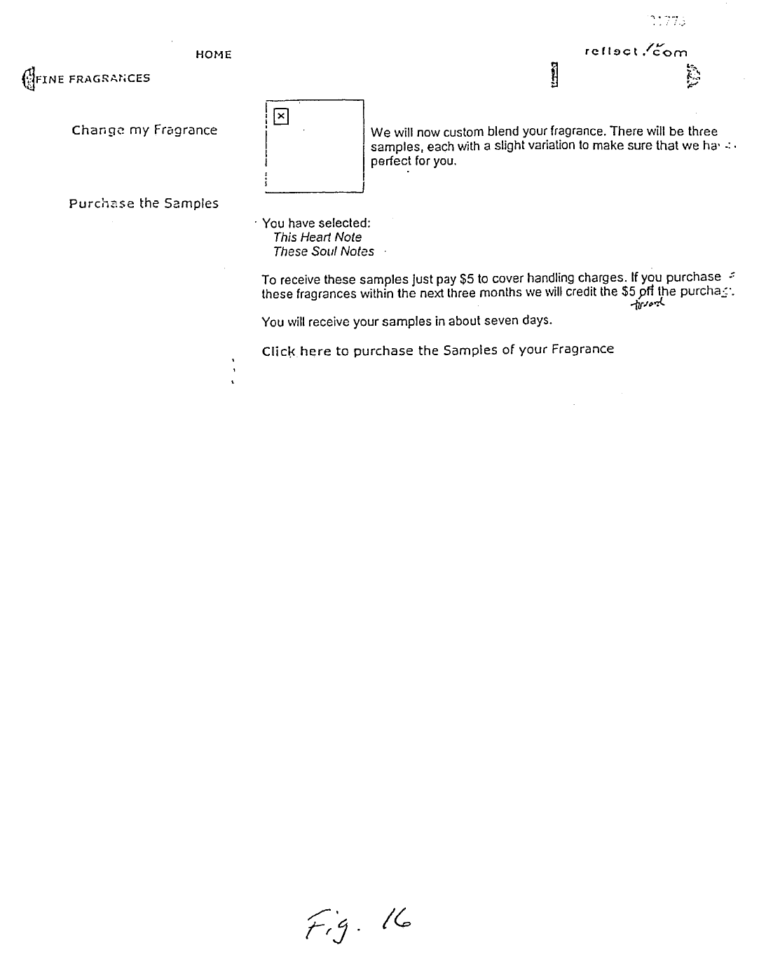

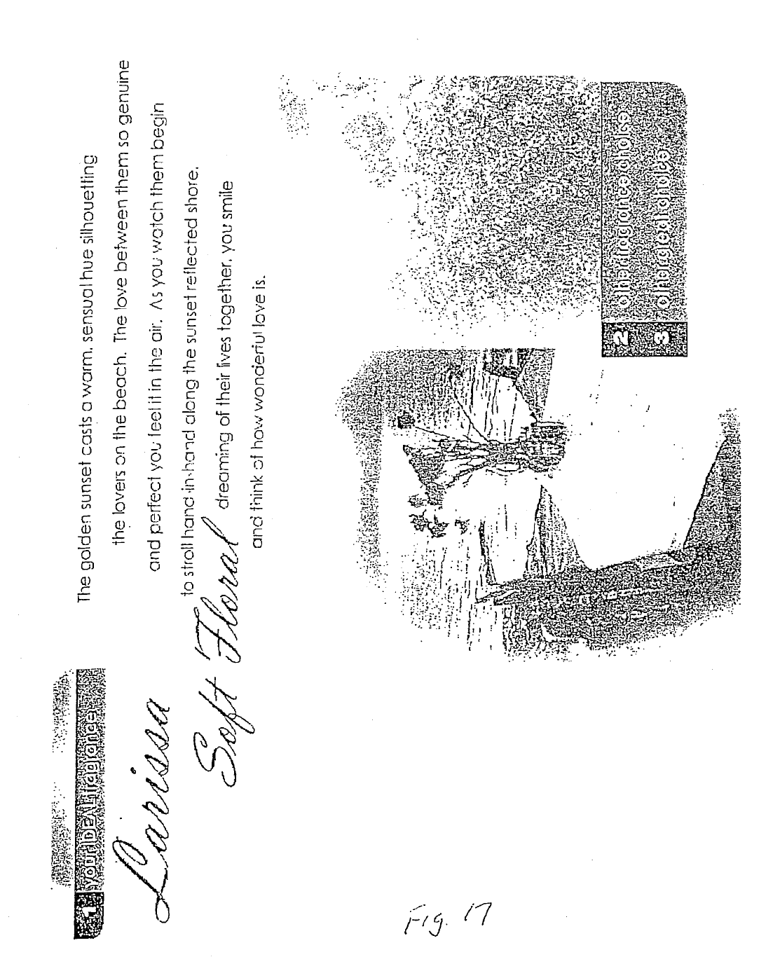

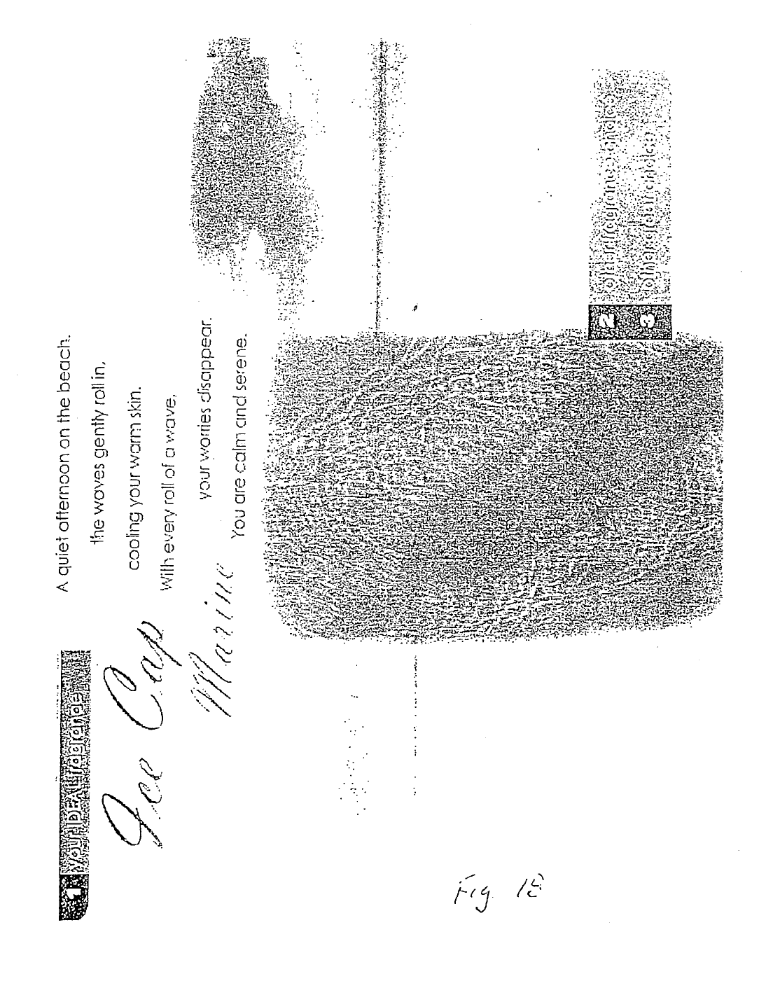

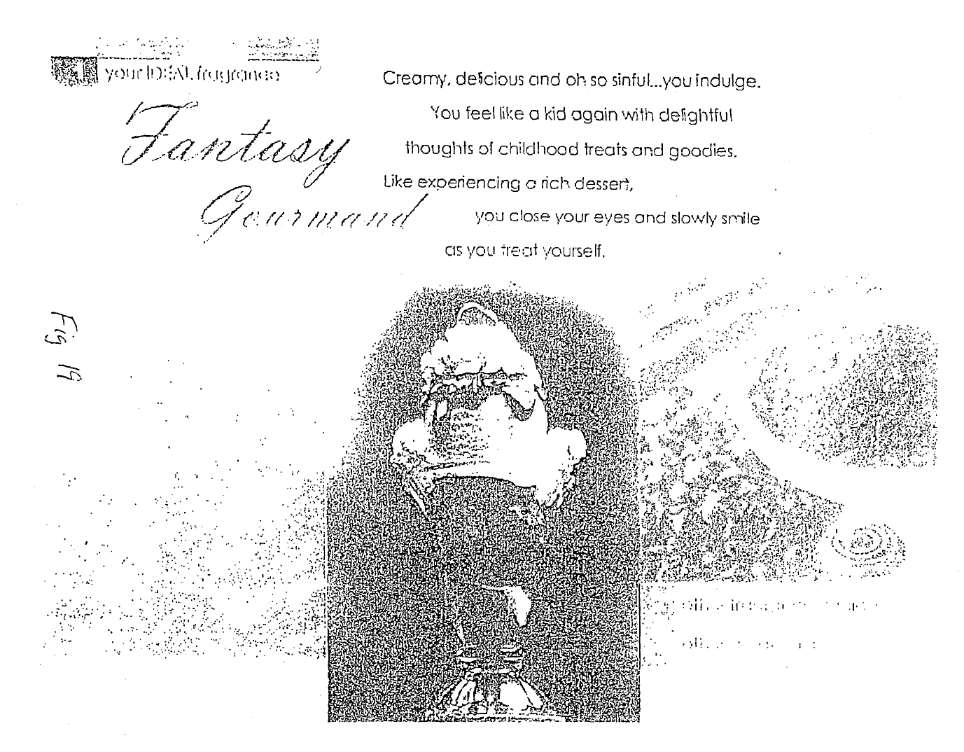

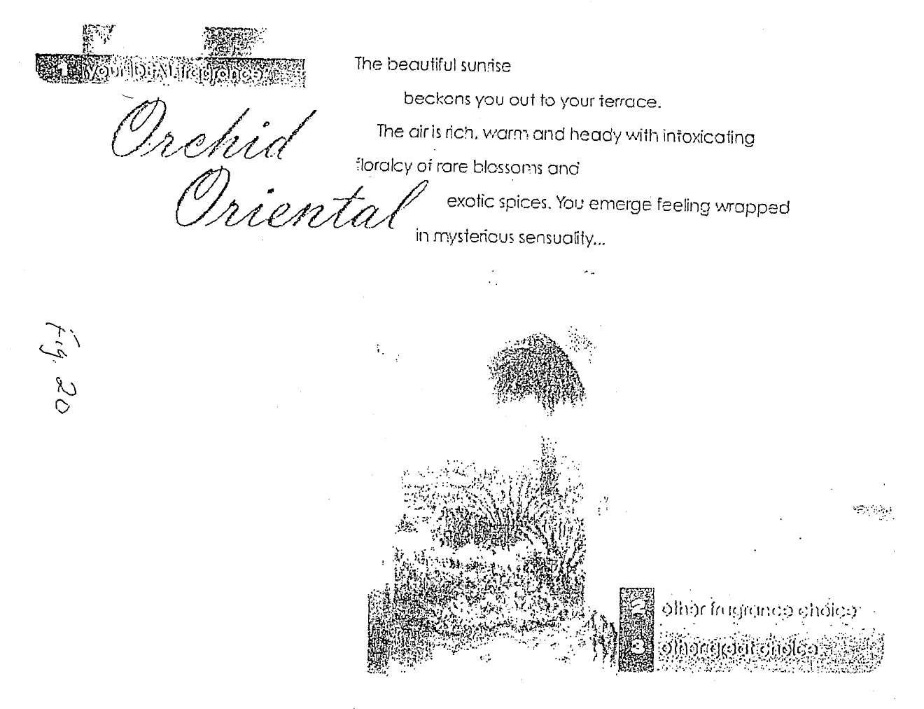

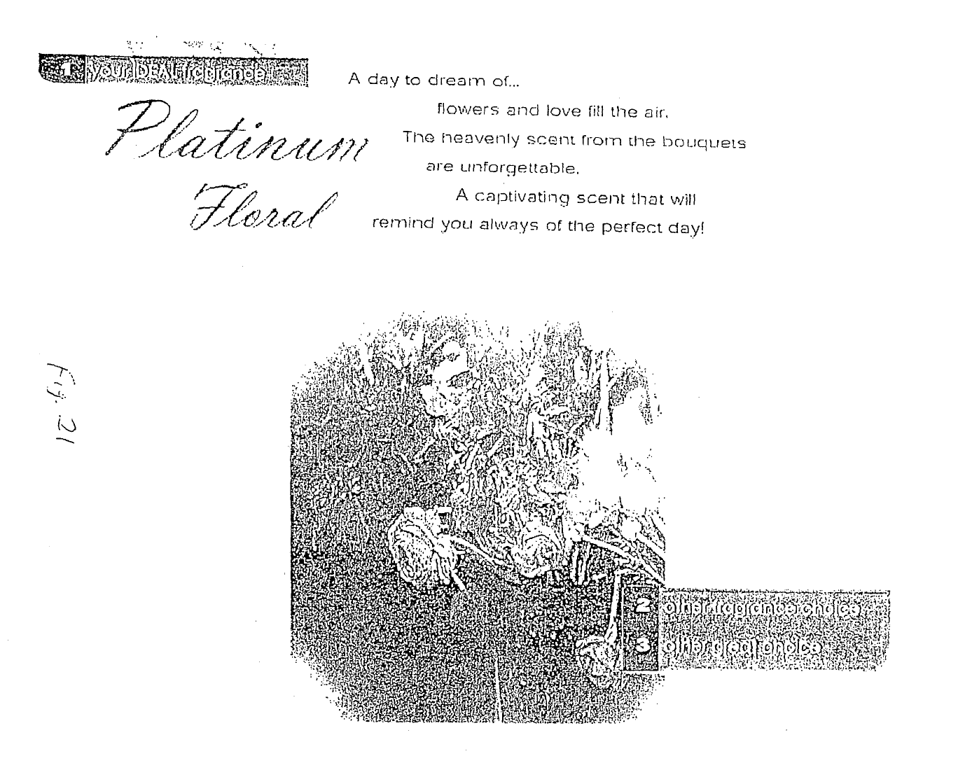

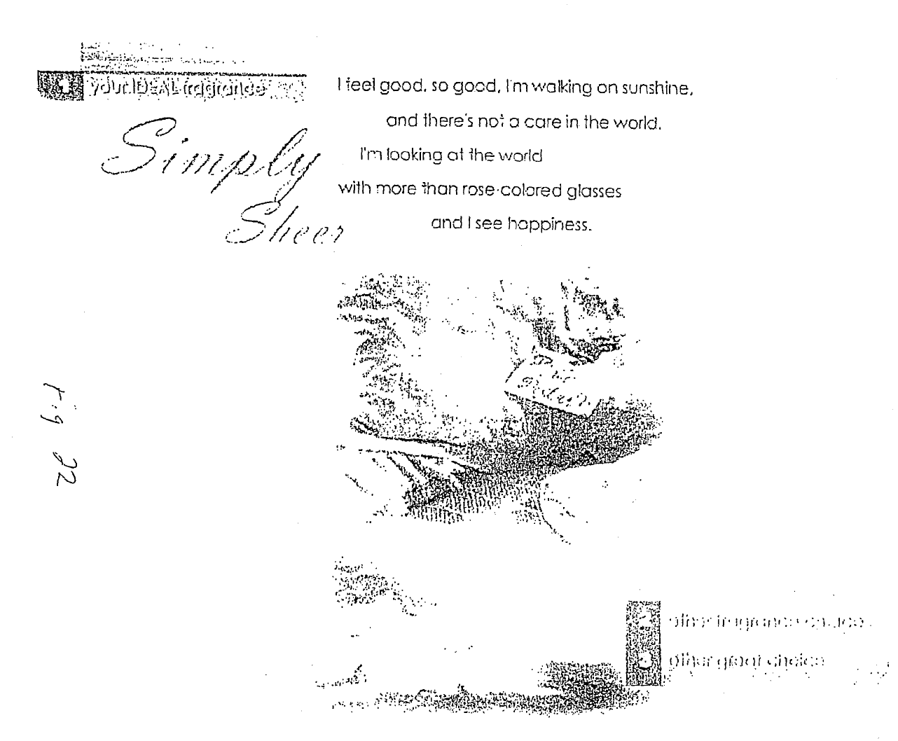

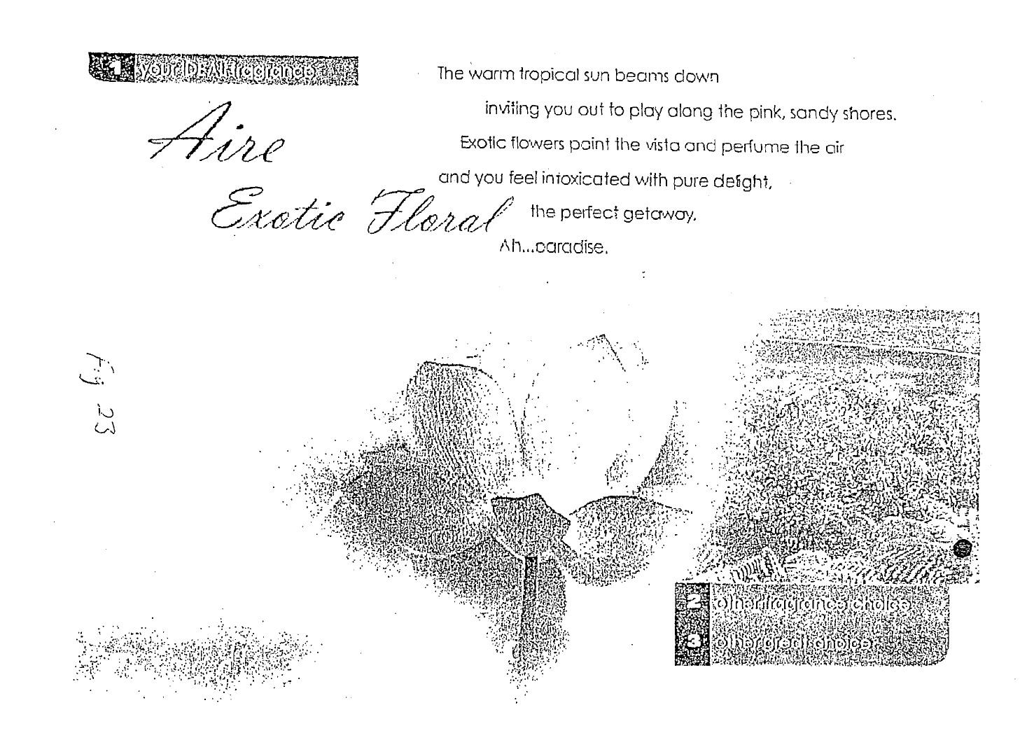

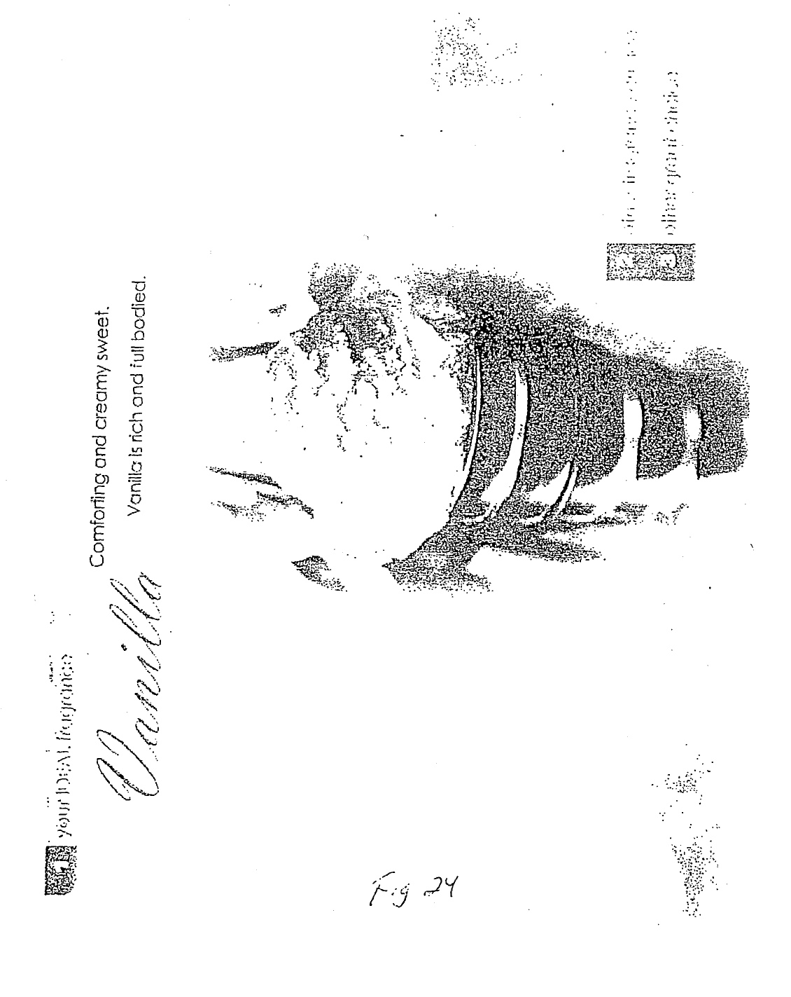

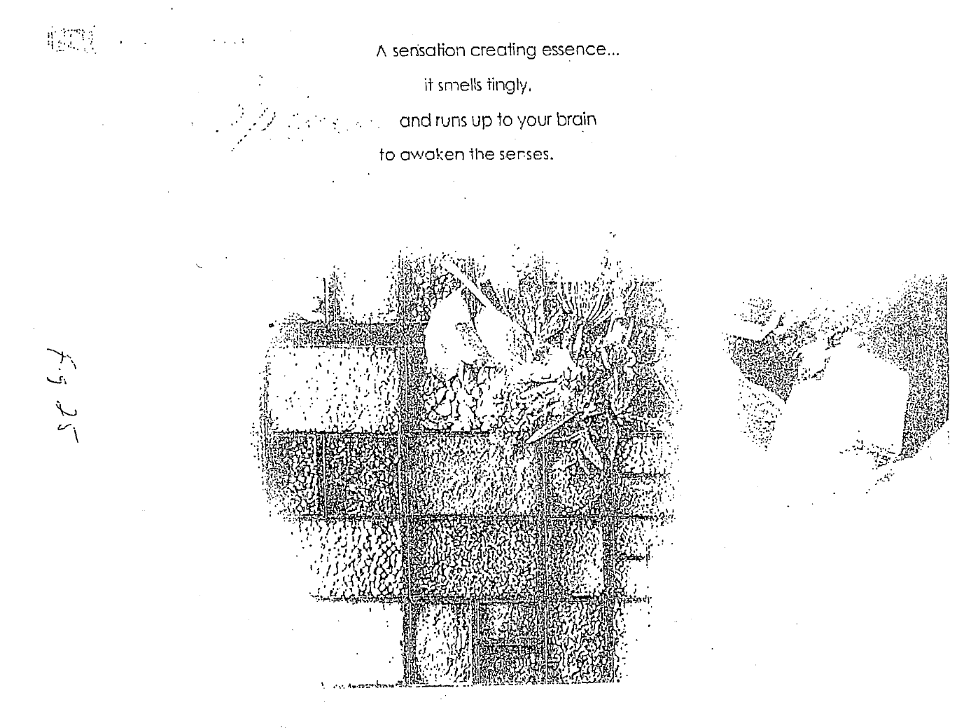

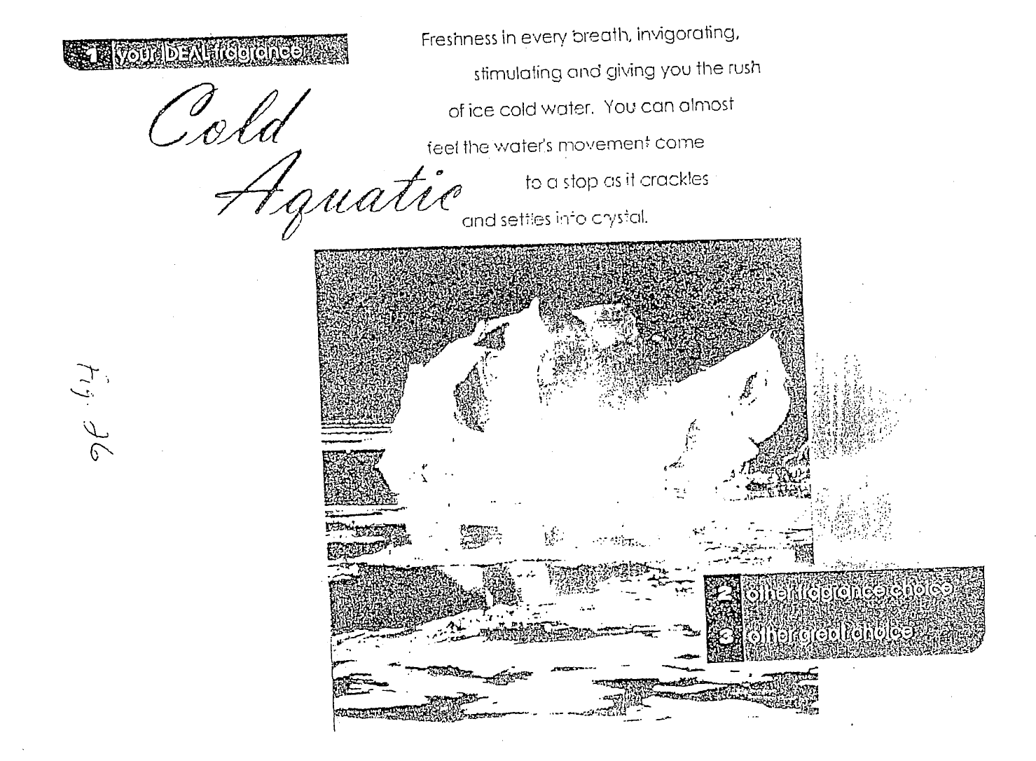

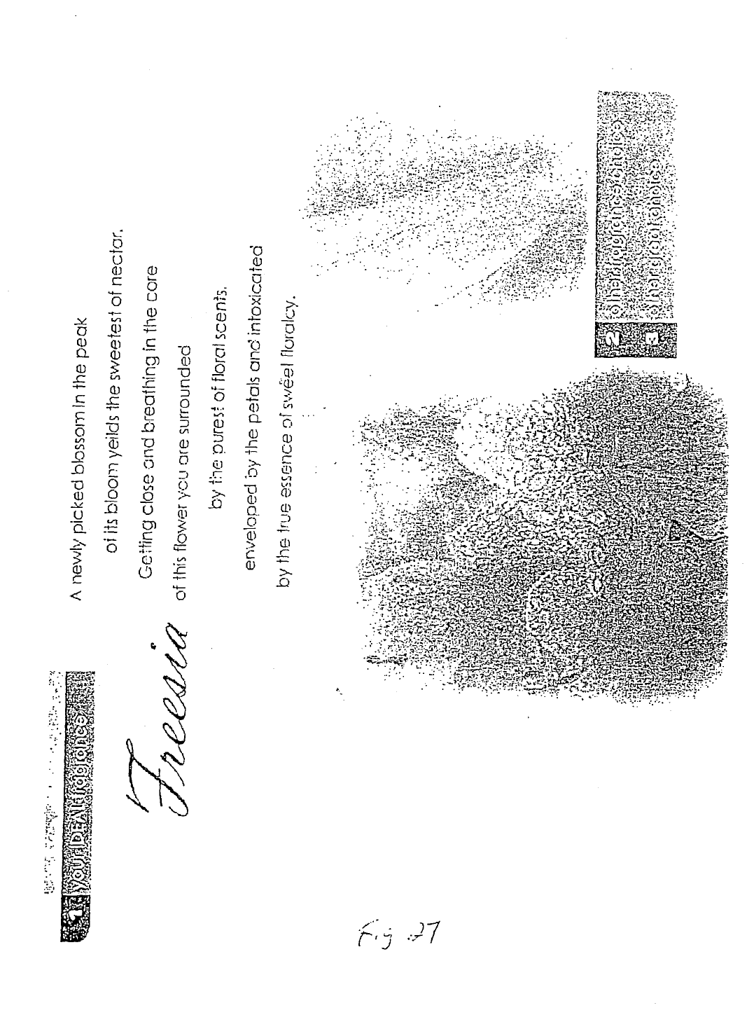

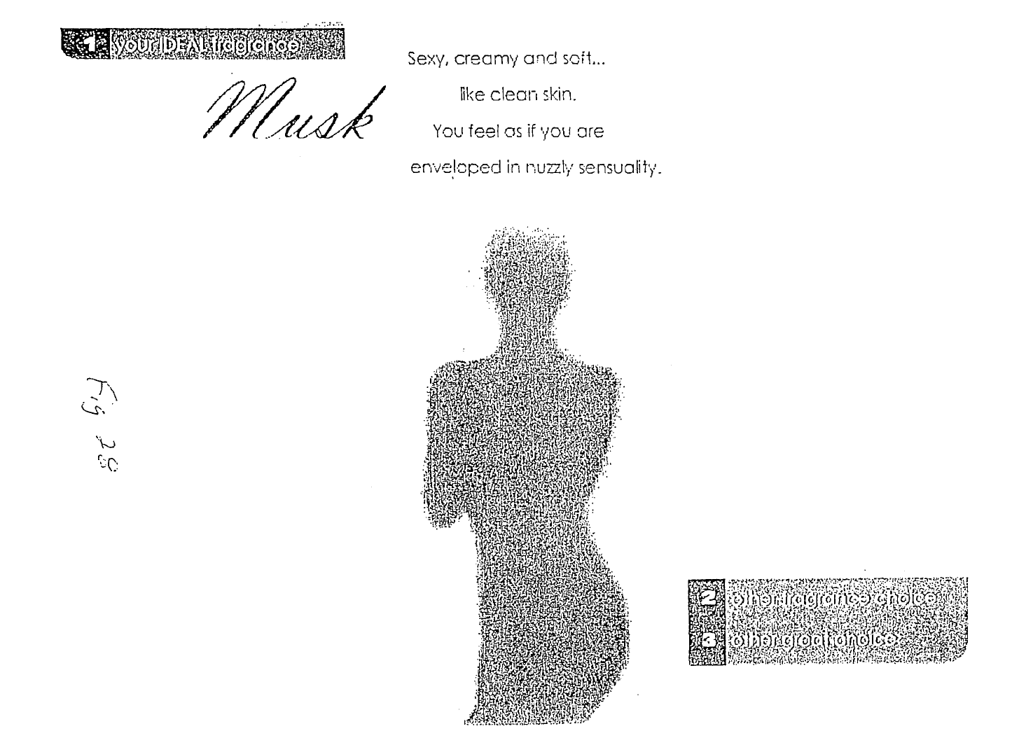

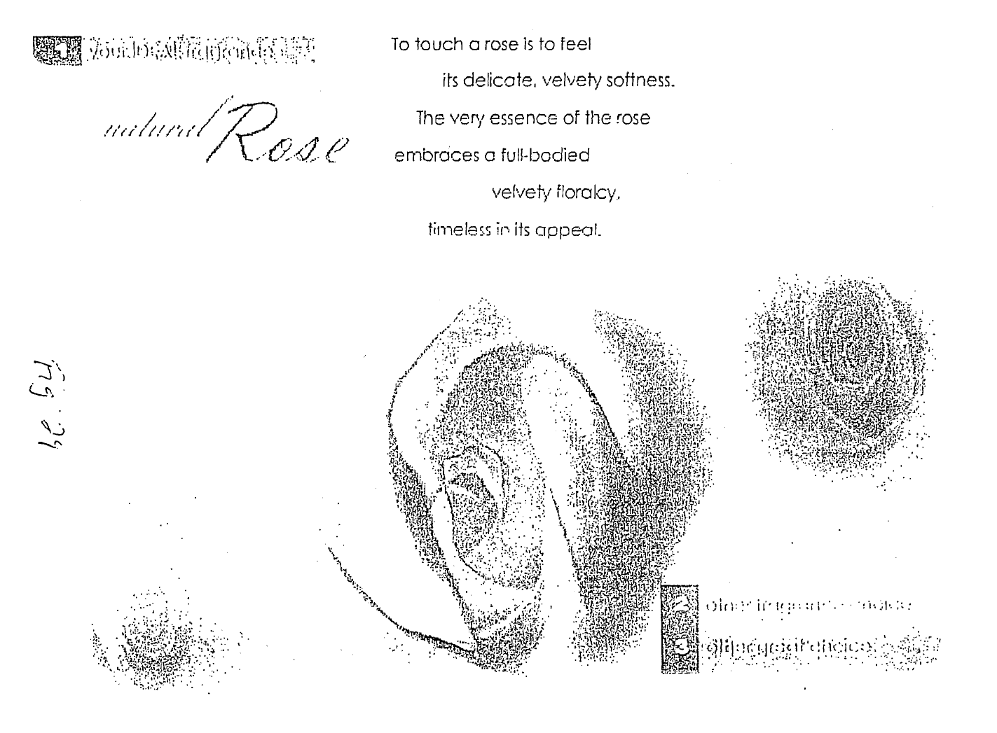

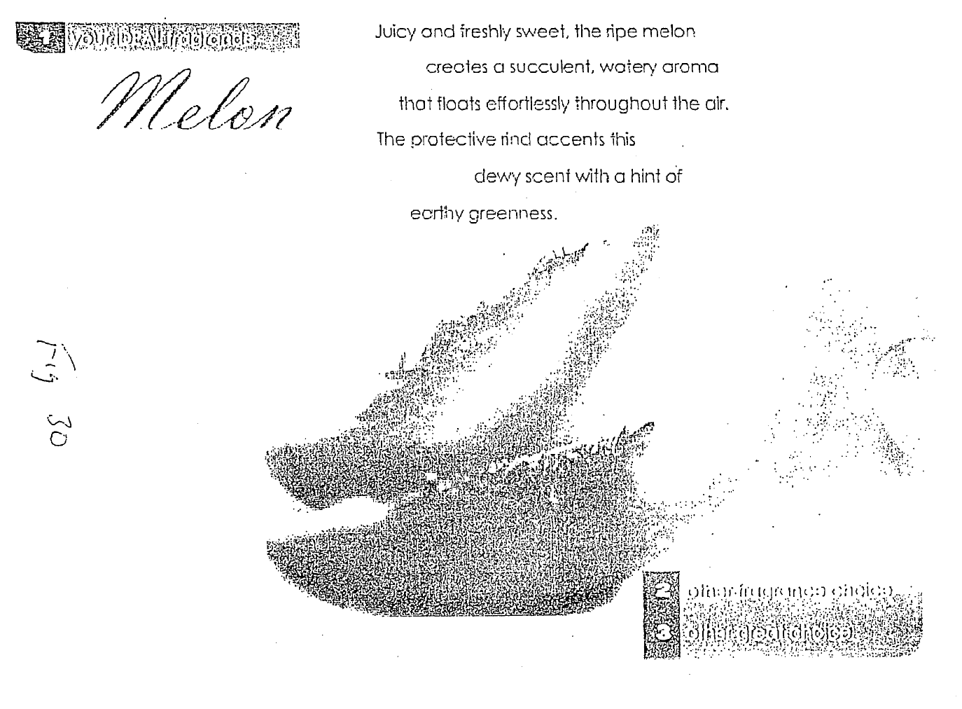

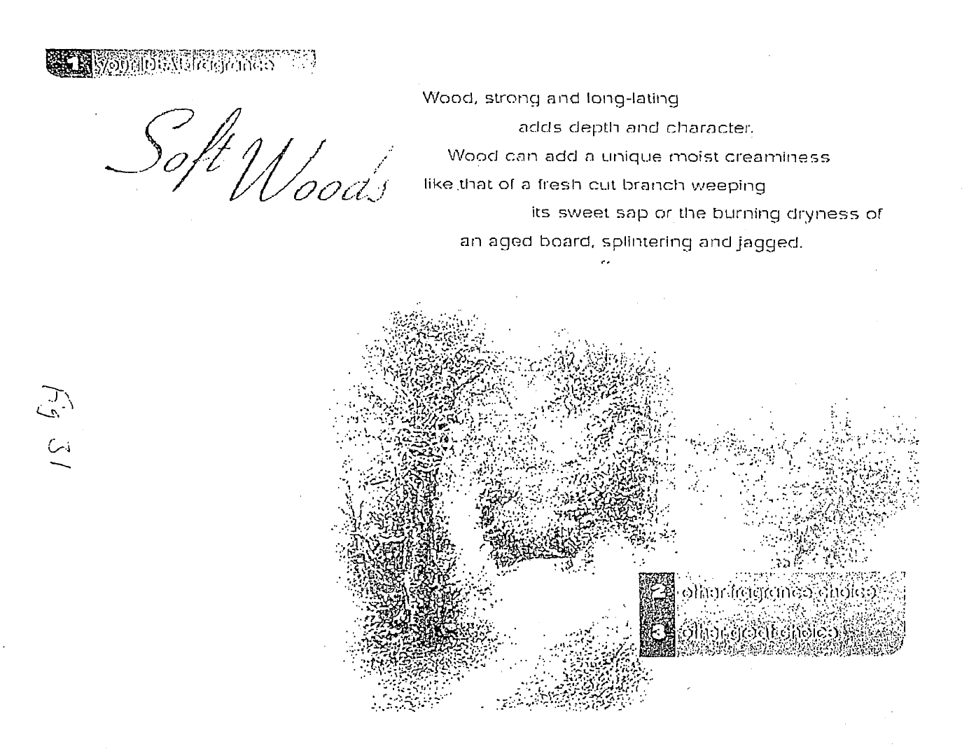

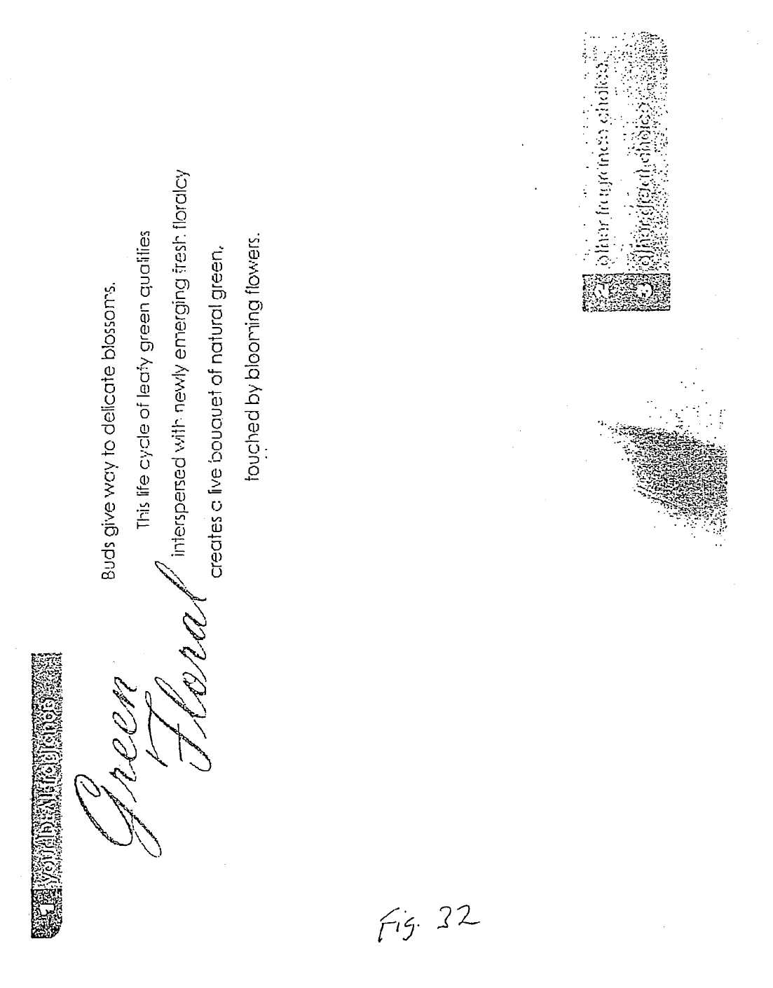

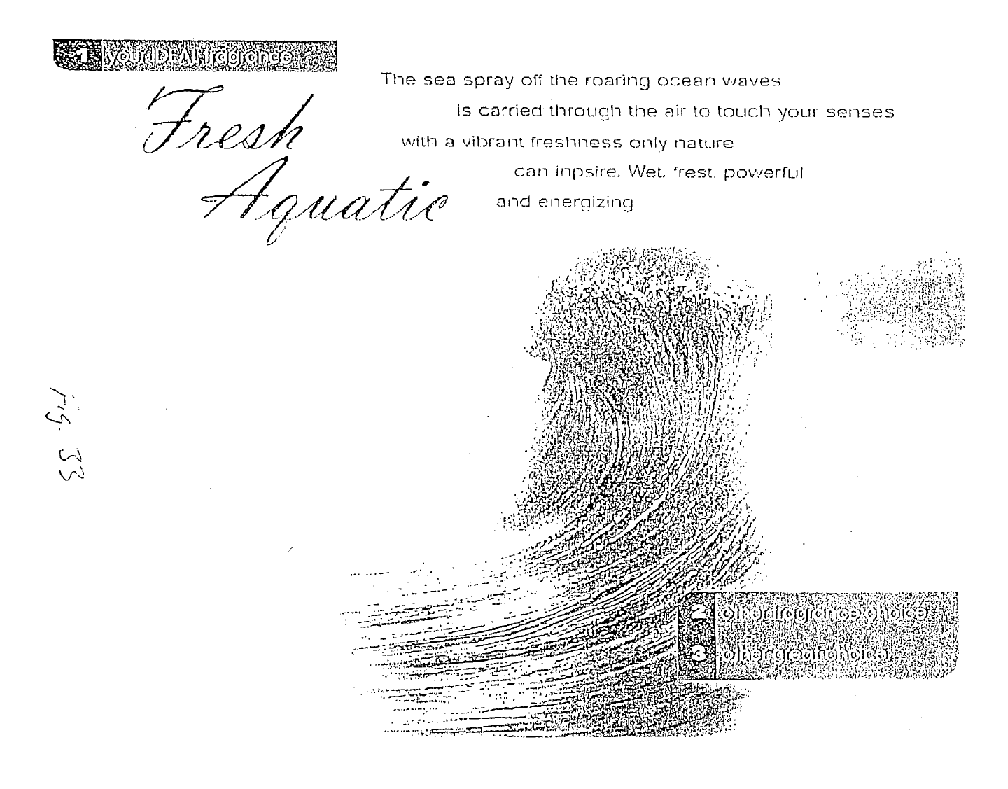

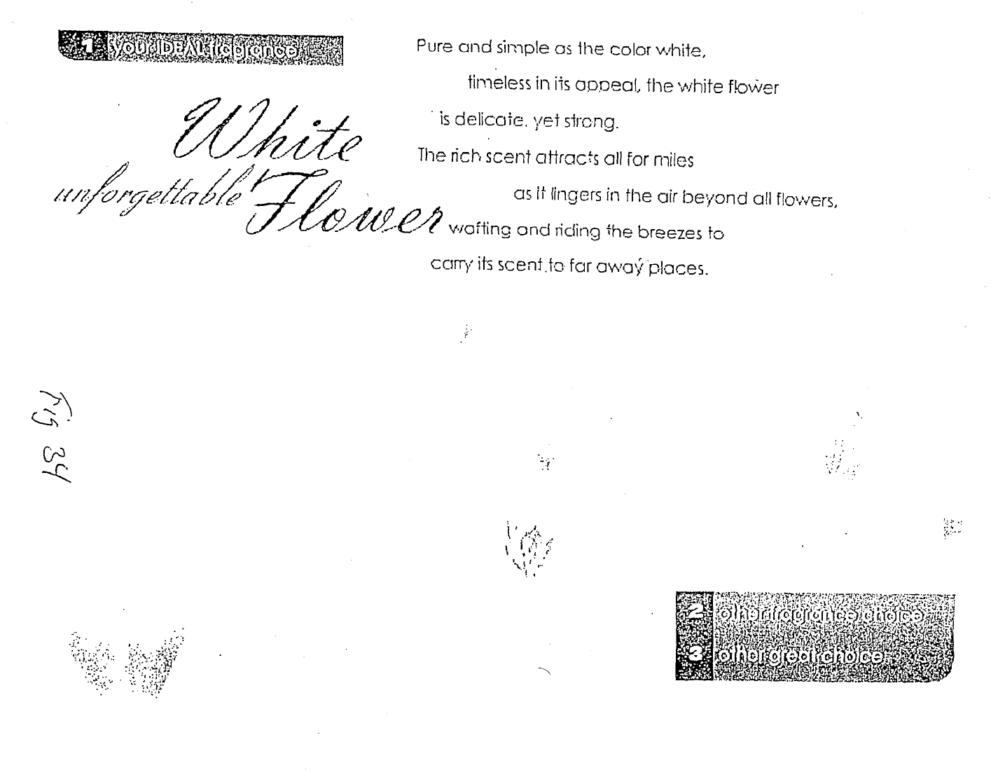

In one embodiment of the present invention, the selection of a customized of fragrance is facilitated by displaying imagery consistent with the time, mood, setting, market trend, and occasion selected by the consumer in response to the questions described above. For example, during the selection of a heart 150, the consumer may select a casual, modern setting during the daytime. If such a selection is made, a “marine” fragrance 150 selection is conveyed to the consumer through imagery consistent with such a setting. For example, a daytime beach scene may be portrayed to convey the selections made by the consumer. An example of such imagery is shown in FIG. 12. Additional examples of imagery consistent with consumer fragrance selections are also shown in FIGS. 17-34. Note that in each of these images, use of color, visual images, and visual metaphors all convey a mood and feeling consistent with the first and second fragrance selections. In another embodiment, audio rendering can also be used to convey the same mood and feeling as the images, or used in combination with the images, each being consistent with a fragrance selection. In this manner, the present invention uses these visual and audio triggers to convey to the consumer a mood/feeling consistent with a fragrance selection.

In one embodiment, once the consumer responds to each of the queries described above, the present invention matches the queries to the available heart fragrances 150. The present invention identifies three (or a lesser or greater number) heart fragrances 150 that most closely match the consumer's answers to the posed questions. Imagery consistent with these three matched heart fragrances 150 is returned and displayed for the consumer. The consumer may then make a final selection of heart fragrance 150.

A similar process is used for the selection of soul fragrances 160. A series of questions is used to determine a season and evocative setting associated with the consumer's answers. The best three (or a lesser or greater number) soul fragrances 160 that most closely match the consumer's answers are identified. Imagery consistent with these three matched soul fragrances 160 is returned and displayed for the consumer. The consumer may then make a final selection of soul fragrance 160.

Having identified the consumer selected heart fragrance 150 and the consumer selected soul fragrance 160, the combined user-customized fragrance is thus identified. The resulting customized fragrance is the combination of the selected heart 150 and the selected soul 160. As described above, the combined fragrance has a high probability of being an appealing fragrance and can be further customized or optimized to the particular consumer's wishes. Needs or desires customized may also be conveyed to the consumer through imagery and audio corresponding to the selected heart 150 and the selected soul 160.

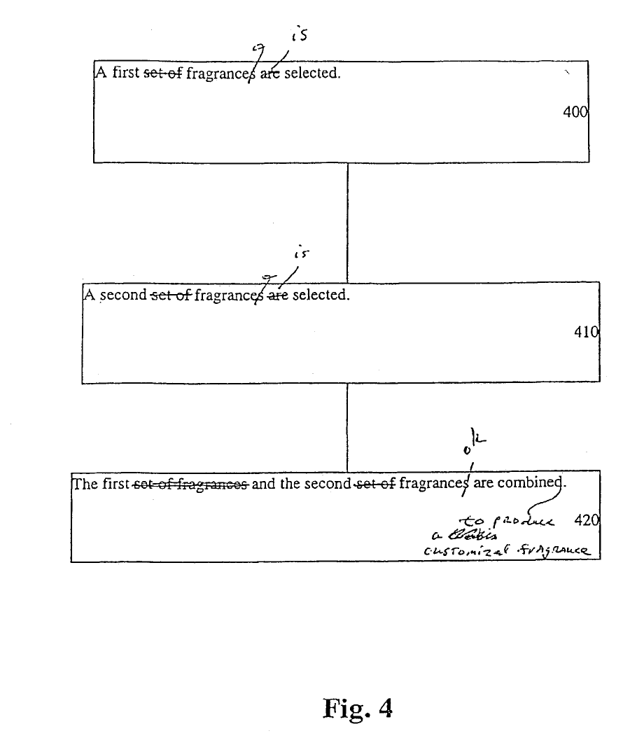

FIG. 4 illustrates a flow diagram of selecting fragrances to form a mixture of fragrances. At block 400, a first fragrances 150 is selected by selecting at least one base note, at least one middle note, and at least one top note. At block 410, a second fragrance 160 is selected by selecting in at least one base note, at least one middle note, and at least one top note. At block 420, the first fragrance 150 and the second fragrance 160 are combined to form a customized fragrance.

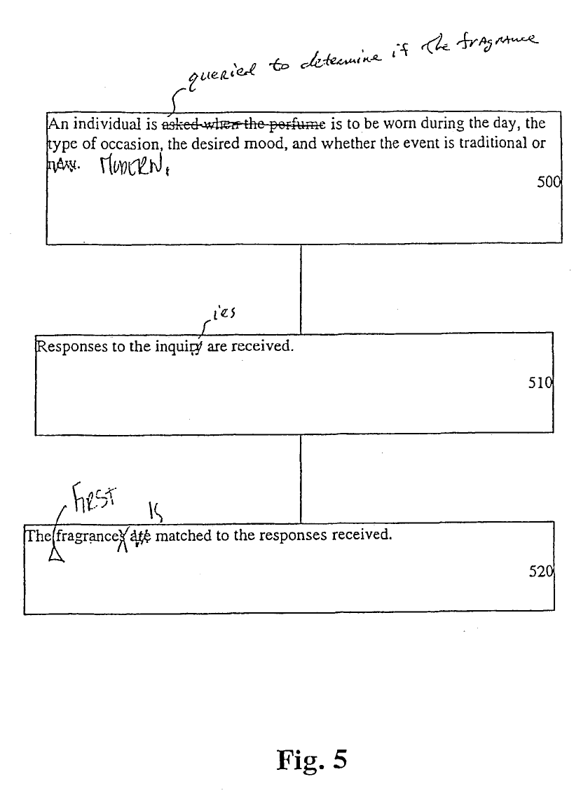

FIG. 5 illustrates a flow diagram of selecting a first fragrance 150 in accordance with one embodiment of the invention. At block 500, an individual is asked for example, when the perfume is to be worn (e.g., during the day), the type of occasion that the perfume is to be worn, the desired mood that the perfume is intended to present, and whether the event is traditional or new. At block 510, responses to these inquiries are received from the individual. At block 520, the first fragrance 150 is matched to the responses received to the questions posed at block 500.

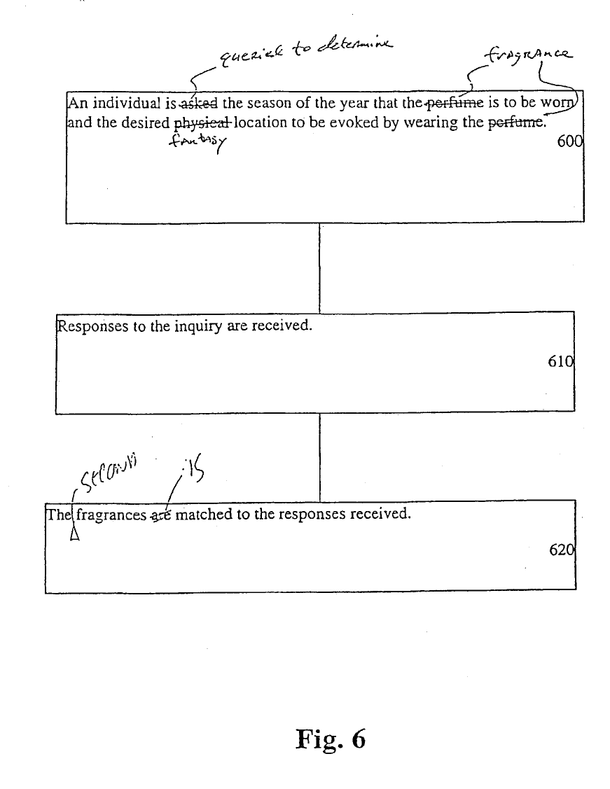

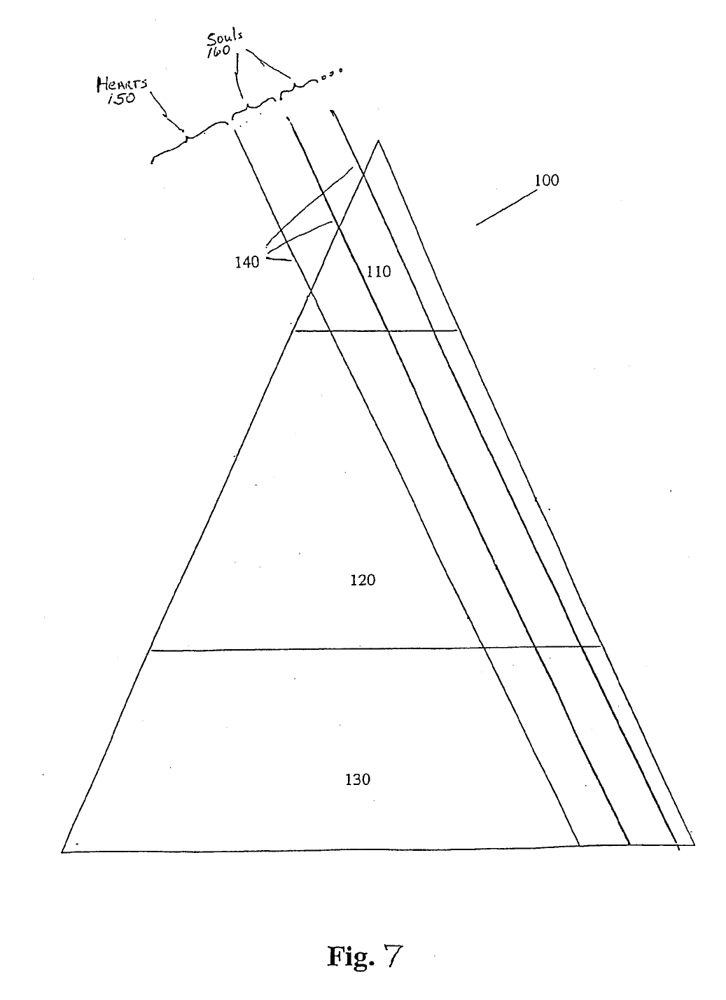

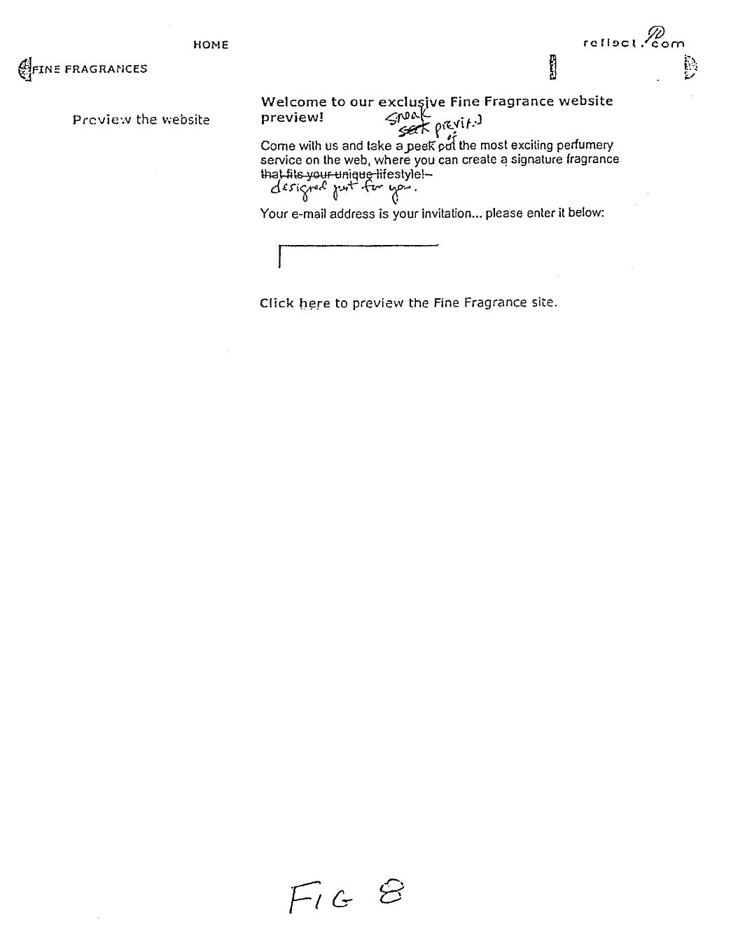

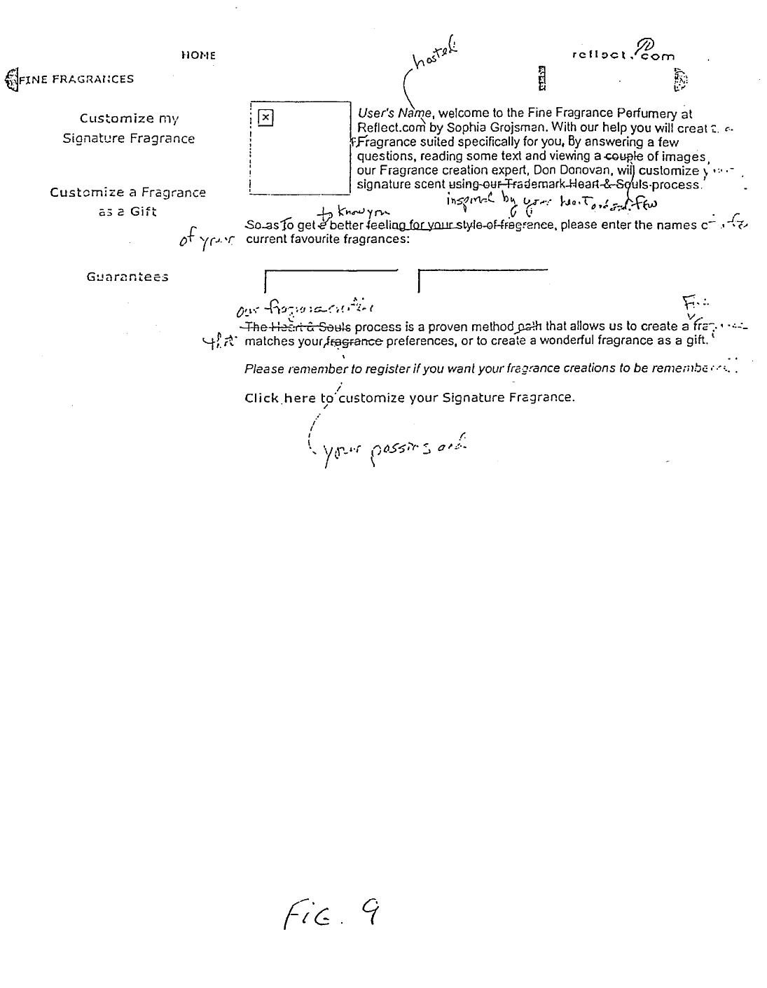

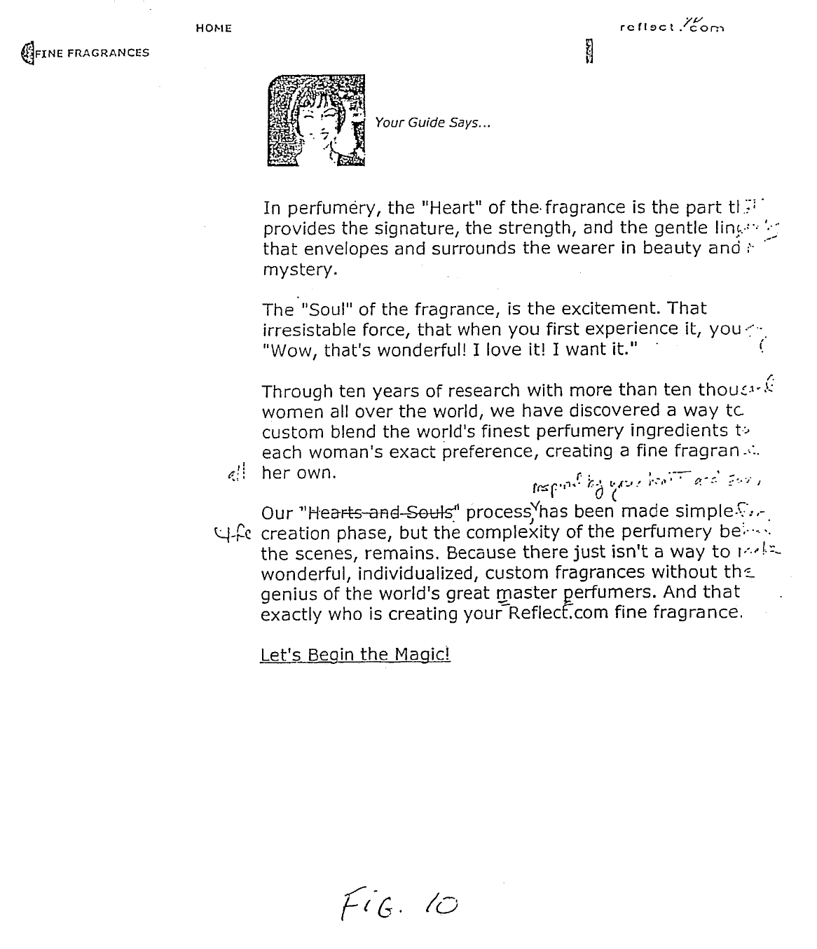

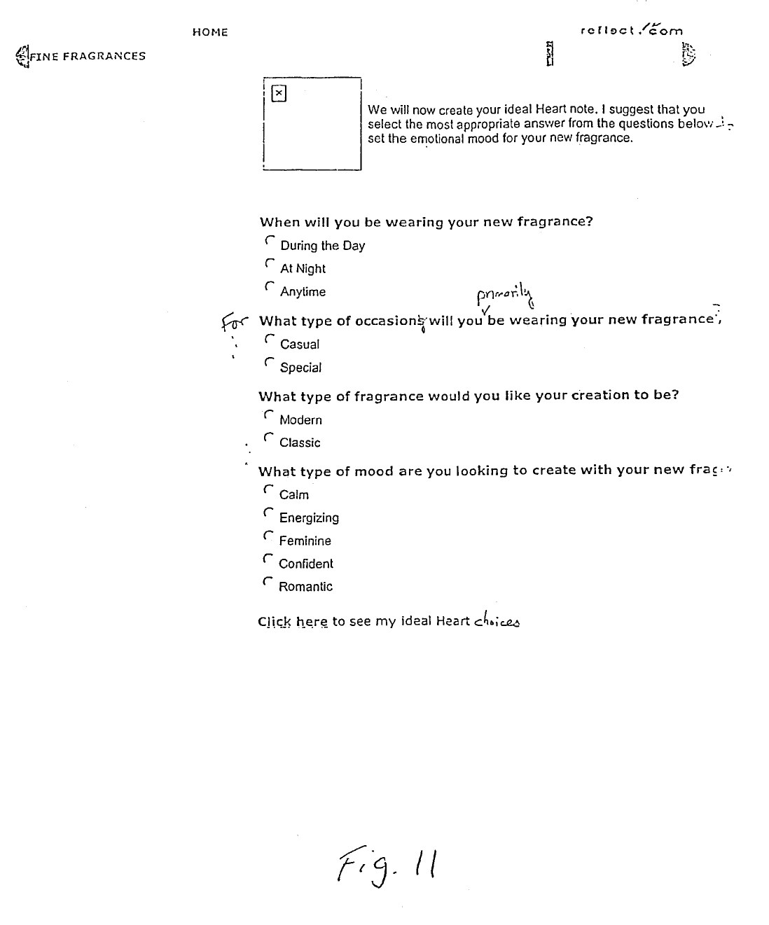

FIG. 6 illustrates a flow diagram illustrating the manner in which the second fragrance 160 is selected. At block 600, questions are posed to the individual as to the season of the year, such as spring, summer, fall, winter, that the perfume is to be worn and the desired physical location to be evoked from wearing the perfume. At block 610, the responses to these questions are received from the individual. At block 620, the second fragrance 160 is matched to the responses received from the individual.

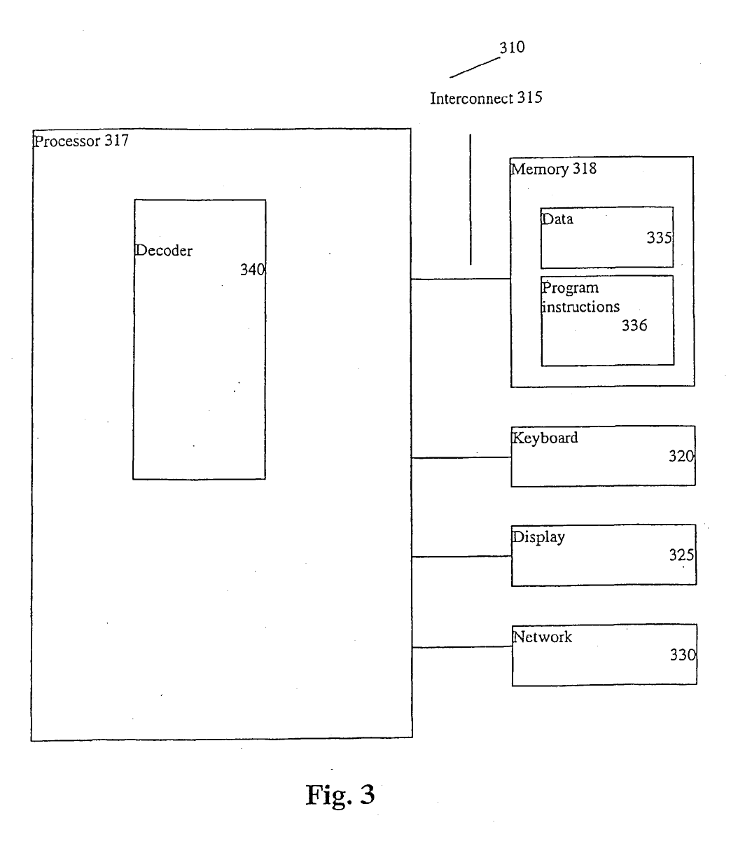

In one embodiment, the process of enabling a user to make selections from a set of hearts 150 and a set of souls 160 to produce a customized fragrance can be implemented on a computer network, such as the Internet. It is important to note that the present invention can be equivalently implemented without the use of computers. In a computer-implemented embodiment, FIG. 3 illustrates one embodiment of a computer system 310, which implements the principles of the present invention. Computer system 310 comprises a processor 317, a storage device 318, and interconnect 315 such as bus or a point-to-point link. Processor 317 is coupled to the storage device 318 by interconnect 315. In addition, a number of user input/output devices, such as a keyboard 320 and a display 325, are coupled to an interface chip set (not shown) which is then connected to processor 317. The interface chipset (not shown) is typically connected to processor 317 using an interconnect that is different from interconnect 315.

Processor 317 represents a central processing unit of any type of architecture (e.g., the Intel architecture, Hewlett Packard architecture, Sun Microsystems architecture, IBM architecture, etc.), or hybrid architecture. In addition, processor 317 could be implemented on one or more semiconductor chips. Storage device 318 represents one or more mechanisms for storing data such as responses to questions posed to an individual. Storage device 318 may include read only memory (ROM), random access memory (RAM), magnetic disk storage media, optical storage media, flash memory devices, and/or other machine-readable media. Interconnect 315 represents one or more buses (e.g., accelerated graphics port bus, peripheral component interconnect bus, industry standard architecture bus, X-Bus, video electronics standards association related to buses, etc.) and bridges (also termed as bus controllers).

While this embodiment is described in relation to a single processor computer system, the invention could be implemented in a multi-processor computer system. In addition to other devices, one or more of a network 330 may be present. Network 330 represents one or more network connections for transmitting data over a machine readable media. The invention could also be implemented on multiple computers connected via such a network.

FIG. 3 also illustrates that the storage device 318 has stored therein data 335 and program instructions (e.g. software, computer program, etc.) 336. Data 335 represents data stored in one or more of the formats described herein. Program instructions 336 represents the necessary code for performing any and/or all of the techniques described herein. It will be recognized by one of ordinary skill in the art that the storage device 318 preferably contains additional software (not shown), which is not necessary to understanding the invention.

FIG. 3 additionally illustrates that the processor 317 includes microprocessor pipeline 340. Microprocessor pipeline 340 may be a program instruction pipeline (e.g. software, computer program, etc.) or a hardware pipeline. Microprocessor pipeline 340 is used for decoding instructions received by processor 317 into control signals and/or microcode entry points. In response to these control signals and/or microcode entry points, microprocessor pipeline 340 performs the appropriate operations.

In the preceding detailed description, the invention is described with reference to specific embodiments thereof. It will, however, be evident that various modifications and changes may be made thereto without departing from the broader spirit and scope of the invention as set forth in the claims. The specification and drawings are, accordingly, to be regarded in an illustrative rather than a restrictive sense.

What is claimed is:

1. A method for preparing a customized fragrance, the method comprising:
selecting a first fragrance, the first fragrance comprising a base note, a middle note and a top note;
selecting a second fragrance, the second fragrance comprising a base note, a middle note and a top note; and
combining the first fragrance with the second fragrance to form the customized fragrance.

2. The method of claim 1, further comprising:
customizing the selection of the first fragrance and the second fragrance based upon information received from an individual.

3. The method of claim 1, wherein the selection of at least one fragrance is based upon a time of day, an occasion, and a desired mood.

4. The method of claim 1, wherein the selection of at least one fragrance is based upon a fantasy location desired to be evoked and a season of a year.

5. A method comprising:
defining a first base-note-dominant fragrance including a top note, a middle note, and a base note;
defining a second top-note-dominant fragrance including a top note, a middle note, and a base note; and
combining said first base-note-dominant fragrance with said second top-note-dominant fragrance produce a composite fragrance.

6. The method of claim 5, further including:
receiving customization information associated with a consumer; and
using the customization information to select the first base-note-dominant fragrance and said second top-note-dominant fragrance to combine into the composite fragrance.

7. The method comprising:
receiving customization information associated with a consumer;
selecting a set of images based upon the customization information, the set of images being associated with a first fragrance comprising a base note, a middle note and a top note;
using the selected set of images to identify the associated first fragrance; and
combining the first fragrance into the composite fragrance.

8. An apparatus comprising:
a machine readable storage media including instructions stored thereon which when executed cause a computer system to perform a method including:
(a) selecting a first fragrance, the first fragrance comprising a base note, a middle note and a top note;
(b) selecting a second fragrance, the second fragrance comprising a base note, a middle note and a top note; and
(c) combining the first fragrance with the second fragrance to form the customized fragrance. The machine readable storage media of claim 8, further comprising:
customizing the selection of the first fragrance and the second fragrance based upon information received from an individual.

10. The machine readable storage media of claim 8, wherein the selection of at least one fragrance is based upon a time of day, an occasion, and a desired mood.

11. The machine readable storage media of claim 8, wherein the selection of at least one fragrance is based upon a fantasy location desired to be evoked and a season of a year.
